(19) United States
(12) Patent Application Publication
(10) Pub. No.: US 2023/0181520 A1
(43) Pub. Date: Jun. 15, 2023

(54) USE OF HYDROPEROXY ALCOHOLS AND THE DERIVATIVES THEREOF AS ANTIMICROBIAL AGENTS

(71) Applicants: DEMETA, RENNES (FR); UNIVERSITE DE RENNES 1, RENNES (FR)

(72) Inventors: FRÉDÉRIC CAIJO, THORIGNE FOUILLARD (FR); CHRISTOPHE DARCEL, RENNES (FR); VINCENT ESCANDE, RENNES (FR)

(21) Appl. No.: 17/779,578

(22) PCT Filed: Nov. 27, 2020
(86) PCT No.: PCT/FR2020/052212

§ 371 Date: Feb. 21, 2023

(30) Foreign Application Priority Data
Nov. 29, 2019 (FR) FR1913526

(51) Int. Cl. A61K 31/357; A61K 31/327; A61Q 5/00; A61Q 7/00; A61K 8/49; A61Q 5/02; A61Q 19/10; A61Q 17/00; A61K 45/06

(52) U.S. Cl. CPC A61K 31/357; A61K 31/327; A61Q 5/006; A61Q 7/00; A61K 8/498; A61Q 5/02; A61Q 19/10; A61Q 17/005; A61K 45/06; A61K 2800/524

(57) ABSTRACT

The invention relates to the use of a hydroperoxy alcohol-type compound, or a 1,2,4-trioxane compound thereof, as an antimicrobial agent, making it possible for it to be used in various applications and in particular as a preservative and/or to improve the antimicrobial properties of at least one preservative, to modulate the skin microbiota, in the treatment or prevention of diseases affecting the skin, the mucous membrane and/or skin appendages or even to cleanse, sanitise, disinfect and/or sterilise a surface other than the skin, skin appendages and mucous membranes, in particular a hard surface or a textile. It also relates to a composition comprising such a compound. In addition to its antimicrobial properties, the aforementioned compound has good safety and emollient properties, making it particularly suitable for cosmetic use.

### SUBJECT OF THE INVENTION

The invention relates to the use of a hydroperoxy alcohol compound, or a 1,2,4-trioxane thereof, as an antimicrobial agent, making it possible to envision its use in various applications and in particular as a preservative and/or for improving the antimicrobial properties of at least one preservative, for modulating the skin microbiota, in the treatment or prevention of a disease affecting the skin, the mucous membrane and/or the skin appendages, or else for cleansing, sanitizing, disinfecting and/or sterilizing a surface other than the skin, the skin appendages and the mucous membranes, in particular a hard surface or a textile. It also relates to a composition, in particular a cosmetic or dermatological composition, comprising such a compound.

### BACKGROUND TO THE INVENTION

Ozonides of plant oils have been known since the beginning of the 20^{th} century. The first processes used oxygen (U.S. Pat. No. 984,722) and then, since the middle of the 1980s, the processes have been based on ozone (U.S. Pat. No. 4,591,602), this gas making it possible to obtain a higher peroxide value and which thus confers, on the ozonated oils obtained, antibacterial, antifungal, antiviral, anti-inflammatory, analgesic and immunomodulatory properties in particular for infections of the skin and the mucous membranes (Koech, Afr. J. Health Sci. 2008, 15, 1). Subsequently, research has been carried out to improve the stability of these ozonides and solutions based on ultrasound (RU 2131673C) or on pulsed electromagnetic fields (WO 2012/168770 A1) have been used while at the same time preserving the antimicrobial properties of the mixtures obtained.

The main problem encountered during these ozonization processes is the need to heat the reaction medium, which results in cleavages of the ozonides formed, thus creating impurities. The main drawback of these impurities is that they decrease the stability and the antibacterial properties of ozonides from plant oils over time. Recourse to iron sulfate-based catalytic processes (WO 2007/046122) admittedly makes it possible to accelerate the process, but not to preserve the stability of the active compounds. Furthermore, the production of these ozonides is highly dependent on the quality of the ozone used and therefore of the ozone generators that are used during these preparations (Travagli, and al. Mediators of Inflammation, Volume 2010, Article ID 610418, 9).

The cleavage of these ozonides also has the effect of decreasing the peroxide value; in point of fact, a very close link between this value and the antimicrobial activity has been established (Diaz, and al. J. Braz. Chem. Soc. 2006, 17, 403).

In addition, ozonated oils often have the reputation of giving off an unpleasant ozone odor. Benzoyl peroxide is, for its part, widely used in the treatment of acne, but this product has the drawback of being irritant to the skin.

There is therefore a real need to have available products which have a high peroxide value, which are well defined and which have a high antimicrobial activity. In addition, there remains the need to have available compounds with good innocuity when they are applied to the skin.

These compounds may be used either as preservatives in various types of products, in particular cosmetic products, or as antimicrobial disinfectants, or even for modulating the skin microbiota.

The skin microbiota is composed of around one million microbes per cm² and is thus thought to consist of several hundreds of distinct species including bacteria, fungi, parasites and viruses. It comprises in particular the transient flora formed from microorganisms that are found more or less sustainably on the skin, depending on the environmental conditions (humidity, pH, temperature) and on the subject's state of health. These microorganisms are generally harmless, but some can cause diseases. Their development is blocked by the microorganisms of the commensal flora (or resident flora) which populate the horny layer and the superficial layer of the epidermis and which live off their host without causing damage to the latter. The commensal flora forms a unique and complex equilibrium, one of the main functions of which is defense of the organism against pathogenic microorganisms. To a certain extent, the term used may be local immunity. By way of example, Staphylococcus epidermidis 5 (Gram+) is one of the most abundant bacteria of the skin microbiota, which helps the skin synthesize antimicrobial peptides that interfere with the colonization of the skin by Staphylococcus aureus (Gram+), which is a common pathogenic agent.

Microorganisms can be classified according to the two types of flora. The microorganisms of the commensal flora are, for example, Propionibacteriaceae (in particular Cutibacterium acnes, P granulosum and P avidum), Staphylococcaceae (for example, S epidermidis, S hominis, S hameolyticus, S aureus and S simulans), Micrococcaceae, Corynebacteriaceae (for example, C. xerosis, C. jeikeium and C. urealyticum), Moraxellaceae of the Acinetobacter genus, the Malassezia yeast types, gram-negative bacilli of the Acinetobacter genus and viruses of the papillomavius type. The microorganisms of the transient flora are for example Staphylococcaceae (S aureus), Streptococcus, Enterobacteriaceae, Pseudomonadaceae, Bacillaceae of the Bacillus genus (for example, B neisseria), and the yeasts of the Candida type (in particular C albicans and C parapsiloopsis). The skin microbiota, which is specific to each individual, can be subjected to multifactorial variations, depending in particular on the area of the skin involved (underarms, navel, soles of the feet, scalp, back, etc.), on the physicochemical characteristics (moisture, salinity, temperature, pH, sweat, sebum), on the endogenous characteristics (sex of the subject, age, genetic factors) and on exogenous characteristics (use of cosmetic and hygiene products, lifestyle, exposure to the sun, environment). It forms a symbiosis with the skin.

It is essential to maintain the balance of the skin microbiota in order to preserve the protective functions of the skin. When there is an imbalance, or dysbiosis, skin disorders can appear. Regulation of the skin microbiota makes it possible in particular to reinforce the barrier function of the skin, including of the scalp, and to thus improve its moisturization, and to reduce skin irritation and the formation of squames and dandruff.

### SUMMARY OF THE INVENTION

In this context, the inventors have demonstrated that hydroperoxy alcohols (or beta-hydroxy hydroperoxides), and also the 1,2,4-trioxanes obtained from the latter, have an excellent antimicrobial activity. It has been possible to prepare these compounds from monounsaturated or polyunsaturated olefins according to a simple and efficient green process. In particular, this process does not make use of ozone, which remains a dangerous and toxic gas, and the use of which must be perfectly overseen. The conversion of the olefins thus transformed is virtually total, and the hydroperoxy alcohols and 1,2,4-trioxanes obtained have a very high peroxide value (2900 meq/kg for the hydroperoxy alcohol derived from oleic acid triglyceride for example). They are in addition provided in an oily form which gives them emollient properties and are thus better tolerated than the antibacterials normally used in dermatology, such as benzoyl peroxide, and more suited to cosmetic application owing to their perfect innocuity. In the pharmaceutical field, these compounds are also preferable to antibiotics in so far as, unlike the latter, they are not liable to generate resistance.

Thus, the invention relates to the use of a hydroperoxy alcohol compound, or a 1,2,4-trioxane thereof, as a preservative and/or for improving the antimicrobial properties of at least one preservative.

It also relates to the cosmetic use of a hydroperoxy alcohol compound, or a 1,2,4-trioxane thereof, for modulating the skin microbiota.

It also relates to a hydroperoxy alcohol compound, or a 1,2,4-trioxane thereof, for use thereof as an antimicrobial agent in the treatment or prevention of a disease affecting the skin, the mucous membrane and/or the skin appendages of a human or animal subject.

It further relates to the use of a hydroperoxy alcohol compound, or a 1,2,4-trioxane thereof, as an antimicrobial agent for cleansing, sanitizing, disinfecting and/or sterilizing a surface other than the skin, the skin appendages and the mucous membranes, in particular a hard surface or a textile.

Another subject of the invention is a composition containing an effective amount of at least one hydroperoxy alcohol compound, or a 1,2,4-trioxane thereof, and at least one excipient chosen from surfactants, thickeners, pigments, dyes, fragrances and mixtures thereof, in particular a cosmetic or dermatological composition also comprising a physiologically acceptable medium optionally containing water.

### FIGURES

FIG. 1 shows the antibacterial activity of the hydroperoxy alcohol of oleic acid on various bacterial strains.

### DETAILED DESCRIPTION

The term “halogen” is intended to mean fluorine, chlorine, bromine or iodine.

The term “alkyl” is intended to mean a linear or branched, saturated or unsaturated, aliphatic hydrocarbon-based group. A “C₁-C₈ alkyl” has from 1 to 8 carbon atoms. Examples of alkyl (or C₁-C₈ alkyl) are in particular a methyl, ethyl, propyl, isopropyl, butyl, isobutyl, tert-butyl, pentyl, hexyl, heptyl or octyl.

The term “aryl” is intended to mean a monocyclic or polycyclic carbocyclic group having from 6 to 20 ring members, containing conjugated double bonds. Examples of aryl groups are phenyl, biphenyl, 1-naphthyl and 2-naphthyl, without this list being limiting.

The term “carbocycle” is intended to mean an aliphatic or aromatic, optionally unsaturated, monocyclic or polycyclic hydrocarbon-based group containing from 5 to 20 carbon atoms.

The term “hydroperoxy alcohol” (or “hydroperoxy alcohol compound”) is intended to mean a cyclic or acyclic, linear or branched hydrocarbon-based chain comprising at least one unit:

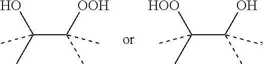

Said hydroperoxy alcohol may be functionalized or nonfunctionalized.

The term “functionalized” is intended to mean that the hydroperoxy alcohol bears at least one, and generally from one to four, inert functional group(s) under the conditions of the hydroperoxy alcohol decomposition reaction, said group(s) being in particular chosen from: a carboxyl group (—COOH), an alkoxycarbonyl group (—OCOR), a hydroxyl group (—OH), a nitro group (—NO₂), a halogen atom (in particular —Cl or —F), an alkoxy group (—OR), an alkylcarbonyl group (—COR), an amido (—CONH₂, —CONHR or —CONR₂) or amino (—NH₂, —NHR or —NR₂) group and a nitrile group (—CN), where R denotes a hydrocarbon-based group (for example, an alkyl or an aryl) containing from one to eight carbon atoms.

In one particular embodiment, the hydroperoxy alcohol compound has the formula (Ia) and/or (Ib):

R¹—CR⁴(OOH)—CR³(OH)—R² (Ia)

R²—CR³(OOH)—CR⁴(OH)—R¹ (Ib)

wherein:

R¹, R², R³ and R⁴ each independently represent a hydrogen atom or a saturated, monounsaturated or polyunsaturated alkyl group or an aryl group, which is optionally substituted, containing from 1 to 30 carbon atoms or a group —L—A where L is a bond or an optionally substituted, linear or branched alkylene chain containing from 1 to 30 carbon atoms, and A represents a hydrogen atom or a group —COXR′ wherein X denotes an oxygen atom or a group —NR″, and R′ and R″ each independently denote a group chosen from: a hydrogen atom, a C₁-C₈ alkyl group, a C₄-C₈ aryl group or a —(CH₂)—CH(OCOR⁵)—CHOCOR⁶ group, R⁵ and R⁶ each independently denoting a linear or branched C₈-C₂₂ alkyl group which is optionally substituted and/or interrupted with at least one hydroperoxy and/or hydroxyl group,

or else R¹ and R², or R³ and R⁴, or R¹ and R⁴, or R² and R³ together form an optionally substituted carbocycle consisting of from 6 to 12 ring members.

The term “1,2,4-trioxane” is intended to mean a hydroperoxy alcohol compound as defined above, of which the —OH and/or —O—OH functions of the hydroperoxy alcohol unit are substituted with a common hydrocarbon-based group, thus forming the following cyclic structure:

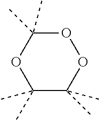

In one particular embodiment, said 1,2,4-trioxane has the formula (IIa) and/or (Ib):

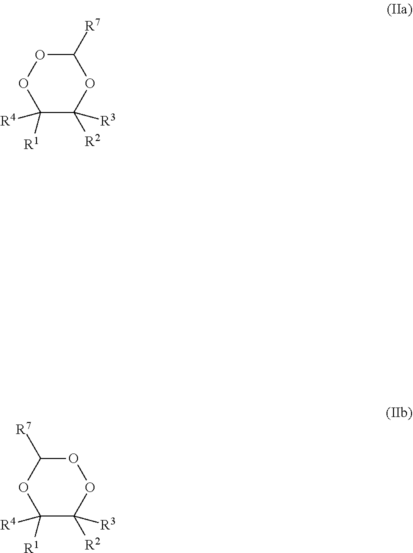

wherein:

R¹, R²R³ and R⁴ each independently represent a hydrogen atom or a saturated, monounsaturated or polyunsaturated alkyl group or an aryl group, which is optionally substituted, containing from 1 to 30 carbon atoms or a group —L—A where L is a bond or an optionally substituted, linear or branched alkylene chain containing from 1 to 30 carbon atoms, and A represents a hydrogen atom or a group —COXR′ wherein X denotes an oxygen atom or a group —NR″, and R′ and R″ each independently denote a group chosen from: a hydrogen atom, a C₁-C₈ alkyl group, a C₄-C₈ aryl group or a —(CH₂)—CH(OCOR⁵)—CHOCOR⁶ group, R⁵ and R⁶ each independently denoting a linear or branched C₈-C₂₂ alkyl group which is optionally substituted and/or interrupted with at least one hydroperoxy and/or hydroxyl group, or else R¹ and R², or R³ and R⁴, or R¹ and R⁴, or R² and R³ together form an optionally substituted carbocycle consisting of from 6 to 12 ring members, and

R⁷ represents a hydrogen atom or a saturated, monounsaturated or polyunsaturated alkyl group or an aryl group, which is optionally substituted, containing from 1 to 30 carbon atoms.

In one particular embodiment, R¹, R², R³ and R⁴ in the compound of formula (Ia), (Ib), (Ha) and/or (IIb) each independently represent a hydrogen atom or an optionally substituted alkyl or aryl group containing from 1 to 30 carbon atoms.

It is preferable for the total number of carbon atoms in R¹, R², R³ and R⁴ to be at least four, more preferentially at least 10, or even at least 14. It is also preferable for at least one of R³ and R⁴ in the compound of formula (Ia), (Ib), (IIa) and/or (Ib) to represent a hydrogen atom.

The alkyl group, when it is substituted, in the compound of formula (Ia), (Ib), (IIa) and/or (Ib), can in particular have at least one (for example, one to four) substituent(s) chosen from a carboxyl group (—COOH), an alkoxycarbonyl group (—OCOR), a hydroxyl group (—OH), a nitro group (—NO₂), a halogen atom (in particular —Cl or —F), an alkoxy group (—OR), an alkylcarbonyl group (—COR), an amido (—CONH₂, —CONHR or —CONR₂) or amino (NH₂, —NHR or —NR₂) group and a nitrile group (—CN), where R denotes a hydrocarbon-based group (for example, an alkyl or an aryl) containing from one to eight carbon atoms.

The hydroperoxy alcohol used according to the invention may be obtained:
directly from the corresponding alkene, or
from the corresponding epoxide, which may optionally itself be obtained from the corresponding alkene.

In one particular embodiment, said hydroperoxy alcohol is obtained by a perhydrolysis process comprising a step of perhydrolysis of the corresponding epoxide by reaction of the epoxide with an aqueous solution of hydrogen peroxide, in the presence of a catalyst consisting of phosphotungstic acid.

In such a perhydrolysis process, the hydrogen peroxide solution is advantageously concentrated to at least 30%, or even at least 45%, and at most 60%, or even 70%, for example to 30%, 45%, 60% or 70% (m/V). Hydrogen peroxide is normally used in an amount ranging from 1 to 1.5 molar equivalent, preferably in a proportion of from 1.1 to 1.3 molar equivalent, relative to the epoxide.

Moreover, in such a perhydrolysis process, the catalyst consisting of phosphotungstic acid is generally used in an amount ranging from 0.01% to 2% by weight, for example of at least 0.1% by weight or of at least 0.2% by weight, in particular of at least 0.4% by weight or of at least 0.5% by weight, and for example of at most 1.5% by weight, in particular of at most 1% by weight, relative to the weight of epoxide. The amount of catalyst consisting of phosphotungstic acid used also represents at least 10 molar ppm, for example at least 50 molar ppm or even at least 100 molar ppm, in particular at least 200 molar ppm, or even at least 400 molar ppm or even at least 800 molar ppm and at most 2000 molar ppm, advantageously at most 1500 molar ppm, or even at most 1000 molar ppm, relative to the molar amount of epoxide.

The perhydrolysis reaction can be carried out at a temperature of from 5 to 60° C., preferably from 20 to 60° C., and more preferentially from 30 to 50° C., for a period ranging from 10 minutes to 4 hours, preferably from 45 minutes to 2 h30. It has been observed that the catalyst consisting of phosphotungstic acid makes it possible to reach a degree of conversion of the epoxide of at least 90%, preferably of at least 95%, or even of at least 99%, after this period of time (as measured by ¹H NMR). The duration of the perhydrolysis reaction is all the shorter the higher the temperature, within the abovementioned ranges.

In such a perhydrolysis process, described in particular in patent FR 3 093 108, the abovementioned reagents (epoxide, hydrogen peroxide and catalyst consisting of phosphotungstic acid) can be introduced in any order into the reactor in which the reaction is carried out. It is moreover preferred for the perhydrolysis reaction to be carried out in the absence of an organic solvent, that is to say of a compound capable of solubilizing the epoxide and/or the catalyst and the structure of which contains one or more carbon atoms. Examples of such solvents are in particular protic polar solvents such as alcohols. It has in fact been demonstrated that these solvents slow down the perhydrolysis reaction at ambient temperature and generally reduce the hydroperoxy alcohol yield.

The perhydrolysis process makes it possible to obtain the desired hydroperoxy alcohol with a yield of at least 70%, generally of at least 80%, indeed of at least 85% or even of at least 90%.

The catalytic constant (TON) of the reaction is moreover always greater than 700 and can range up to more than 15 000.

The epoxide can itself be obtained by epoxidation of the corresponding alkene. The epoxidation can be carried out conventionally for those skilled in the art, for example by means of a percarboxylic acid, in particular performic acid, as described for example in document JP2015-083638.

Alternatively, hydroperoxy alcohol can be obtained directly from the corresponding alkene. Conditions for carrying out such a process are in particular described in patent application JP 2003/342255.

In one particular embodiment, said hydroperoxy alcohol compound or a 1,2,4-trioxane thereof is formed from an alkene (optionally via an epoxide), said alkene being chosen from at least one monounsaturated or polyunsaturated fatty acid or the ester thereof, or mixtures thereof, in particular at least one alkyl ester of said fatty acid or at least one glyceride of said fatty acid, or a terpene.

Examples of monounsaturated or polyunsaturated fatty acids are in particular palmitoleic acid, oleic acid, erucic acid, ricinoleic acid or nervonic acid. In certain cases, the fatty acid or glyceride thereof can itself be derived from a plant oil, and the fatty acid alkyl ester can be obtained by transesterification of at least one plant oil. As examples of plant oils, mention may in particular be made of wheatgerm oil, sunflower oil, argan oil, hibiscus oil, coriander oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, blackcurrant oil, evening primrose oil, lavender oil, borage oil, millet oil, barley oil, quinoa oil, rye oil, safflower oil, candlenut oil, passionflower oil, musk rose oil, Echium oil, camelina oil or camelia oil.

Thus, in one embodiment of the invention, the hydroperoxy alcohol compound is obtained by perhydrolysis of an epoxidized plant oil, advantageously chosen from those listed above, preferably olive oil.

The terpenes comprise in particular monoterpenes, sesquiterpenes, diterpenes, sesterpenes, triterpenes, carotenoids or else terpenoids, preferably sesquiterpenes or carotenoids. Examples of terpenes are in particular α-pinene, β-pinene, carene, limonene, carotene, ocimene, myrcene, citronellene, methoxycitronellene, farnesene, squalene and astaxanthine, without this list being limiting.

Other examples of alkenes are in particular dodecene, tridecene, cycloheptene, cyclooctene, or cycloocta-1,5-diene, without this list being limiting.

There are several methods for preparing a 1,2,4-trioxane from a hydroperoxy alcohol. For example, 1,2,4-trioxane can be prepared by reaction of a hydroperoxy alcohol with an aldehyde or a ketone, generally in the presence of a Brönsted or Lewis acid catalyst. Such a method is in particular described in the article Griesbeck et al. Org. Lett. 2002, 4, 24, 4193-4195.

The compounds described above have proven to be effective as antimicrobial agents, thereby making it possible to envision their use as preservatives, or as cosmetic or dermatological active agents, or else as disinfectant agents, depending on the dose used that those skilled in the art will be able to easily determine.

A first subject of the invention thus relates to the use of a hydroperoxy alcohol compound, or a 1,2,4-trioxane thereof, as a preservative and/or for improving the antimicrobial properties of at least one preservative.

In this application, the compound according to the invention can be formulated in any type of composition, in particular cosmetic, dermatological or detergent composition, provided in particular in the form of an oily composition or of an emulsion. Depending on its use, this composition can be applied to any type of substrate, in particular to the skin, the skin appendages and in particular the hair or the eyelashes, or else to hard surfaces or textiles. Examples of preservatives are 1,2-(C₅-C₁₀ alkane)diols such as 1,3-propanediol, pentylene glycol, hexanediol, octanediol (or caprylyl glycol) and decanediol; ethers of glycerol and of C₃-C₈ alkyl, such as octoxyglycerol; hinokitiol; benzoic acid; sorbic acid; potassium sorbate; dehydroacetic acid; phenoxyethanol; parabens; and mixtures thereof.

For a use as a preservative or preservative booster, it is preferable for the abovementioned composition to contain from 0.001% to 1%, preferably from 0.01% to 0.1%, of hydroperoxy alcohol compound, or a 1,2,4-trioxane thereof.

Another subject of the invention relates to the cosmetic use of a hydroperoxy alcohol compound, or a 1,2,4-trioxane thereof, for modulating the skin microbiota and thus preventing and/or reducing the unattractive manifestations of the detrimental modification of the microbiota of the skin, including of the scalp.

The term “cosmetic use” is intended to mean a nontherapeutic use on healthy skin.

The term “modulating the skin microbiota” is intended to mean preventing and/or treating an imbalance of the skin microbiota that may be due to one or more extrinsic factors, such as environmental factors (temperature, ambient humidity, pollution, UV), drug treatments (antibiotic, corticoid), or else the application the skin of hygiene products (containing in particular fatty acid soaps), of detergents or of antiperspirants. The modulation of the skin microbiota comprises more particularly the modification of the relative proportion of the microorganisms present at the surface of the skin, including optionally the scalp. The modulation of the skin microbiota makes it possible in particular to prevent or reduce at least one unattractive manifestation of the imbalance of the skin microbiota, such as the formation of dandruff and/or of squames (resulting in particular from an imbalance between bacteria and fungi of the Malassezia genus), the formation of blackheads, roughness of the skin, non-uniformities of the complexion, the development of odors (formed in particular by reaction of S hominis with sebum), and/or hair loss.

When it is used for modulating the microbiota of the skin, it is preferable for the hydroperoxy alcohol compound, or a 1,2,4-trioxane thereof, to be included in a cosmetic composition containing from 0.001% to 15%, preferably from 0.01% to 10%, for example from 0.1% to 5% by weight, of this compound.

This cosmetic composition comprises a cosmetically acceptable medium, that is to say a medium suitable for topical application to the skin (including the scalp), the mucous membranes and/or the skin appendages and which does not cause redness, stinging or other unpleasant manifestations incompatible with a cosmetic application. It is in particular in the form of an oily composition or of an emulsion, more preferentially of a water-in-oil or oil-in-water emulsion. The composition can have a more or less fluid texture and can be in the form of an oily solution or dispersion, of a fluid, of a cream, of a balm or of a solid composition of bar type. It can be packaged in a pump-action bottle, a tube, a jar or an aerosol device. The cosmetic composition can for example be used as a composition per se, for cleansing and/or making up the skin, or else as a shampoo or conditioner.

Another subject of the invention relates to a hydroperoxy alcohol compound, or a 1,2,4-trioxane thereof, for use thereof as an antimicrobial agent, in the treatment or prevention of a disease affecting the skin, the mucous membrane and/or the skin appendages of a human or animal subject.

The term “antimicrobial agent” is intended to mean a substance which kills, slows the growth of or blocks the growth of one or more microbes. The term “growth” denotes in the present invention any cell operation which allows a volumetric increase in the cell, a cell division or a cell reproduction. According to the present invention, a microbe denotes any single-cell or multicellular organism which is pathogenic or parasitic with respect to other living organisms such as human beings or animals. Among microbes, mention may be made in particular of molds, fungi, yeast, bacteria and viruses. An antimicrobial agent according to the present invention can for example be an antibiotic, antiviral, antifungal and/or antibacterial agent.

The term “antifungal” is intended to mean in particular fungicidal and/or fungistatic.

The term “antibacterial” is intended to mean in particular bactericidal and/or bacteriostatic.

The term “fungicidal, or fungistatic, agent” denotes an agent capable respectively of eliminating at least one type of mold, fungus or yeast, or of slowing down the development of at least one type of mold, fungus or yeast.

The term “bactericidal or bacteriostatic agent” denotes an agent capable respectively of eliminating at least one type of bacterium or of slowing down the development of at least one type of bacterium.

The term “bacterium” denotes eubacteria and archaebacteria. The eubacteria include the firmicutes, the gracilicutes and the terenicutes. The gracilicutes include Gram-negative bacteria such as the Enterobacteriaceae, for instance Klebsellia (such as Klebsellia pneumoniae) and Escherichia (such as Escherichia coli). The firmicutes include Gram-positive bacteria, such as Micrococcaceae, for instance the Staphylococae (such as Staphylococcus aureus) and the stalks forming endospores include the bacilli (Bacillaceae), for instance Bacillus circulans. All these references are mentioned in Bergey's Manual of Systematic Bacteriology, Williams & Wilkens, 1st ed. Vol. 1-4, (1984).

Preferably, the bacteria are chosen from: Propionibacteriaceae (in particular Cutibacterium acnes, P gramlosum and P aidum), Staphylococcaceae (for example S epidermidis, S hominis, S hameolyticus, S aureus and S simulans), Micrococcaceae, Corynebacteriaceae (for example C. xerosis, C jeikeiur and C urealvticum), Moraxellaceae of the Acinetobacter genus, Streptococcus, Enterobacteriaceae, Pseudomonadaceae, Bacillaceae of the Bacillus genus (for example B neisseria).

The term “molds” denotes in particular fungi and yeast, preferably of the Malassezia genus, in particular, M globosa and M restricta, or of the Candida genus, in particular C albicans and C parapsiloopsis, or else of the Aspergillis genus, such as Aspergillus niger, or else of the Trichophyton or Microsporum genus.

In one particular embodiment, said antimicrobial agent is an antibacterial agent.

In the case of human subjects, examples of diseases affecting the skin, the mucous membrane and/or the skin appendages are in particular acne, herpes, mycosis, athlete's foot, chickenpox, warts, shingles, psoriasis, conjunctivitis, styes, eczema, vitiligo, atopic dermatitis or a keratosis, preferably acne. An example of a disease affecting the skin of an animal subject is ringworm.

The term “treatment” of a disease (or disorder, or condition) comprises the reduction of at least one symptom thereof, a reduction in its seriousness, its delay or its inhibition. The treatment does not necessarily mean that the disease (or the disorder or the condition) is totally cured. A compound used according to the present invention can, in certain cases, only reduce the seriousness of a disease (or disorder, or condition), reduce the seriousness of the symptoms that are associated therewith, improve the quality of life of the patient or of a subject, or delay or inhibit the appearance of a disease (or disorder, or condition).

The term “prevention” denotes any reduction, however little, of the predisposition to or the risk of developing a condition, a disease, a disorder or a symptom thereof. The subject may be any subject and, preferably, is a subject who risks developing or is predisposed to the development of an ailment, a disease or a disorder. The term “prevention” includes either the prevention of the appearance of a disease, or of a disorder that is clinically obvious, or the prevention of the appearance of a disease, or of a disorder that is preclinical, in individuals or animals at risk.

This comprises the prophylactic treatment of the subjects at risk of developing a disease or a disorder. The compounds according to the invention are advantageously used as antimicrobial agents for combatting bacteria of the Staphylococcus, Streptococcus, Corynebacterium and Propionibacterium genera involved in particular in atopic dermatitis, acne and psoriasis.

For a pharmaceutical (including veterinary) application, it is preferable for the hydroperoxy alcohol compound, or a 1,2,4-trioxane thereof, to be included in a composition containing from 0.5% to 10%, preferably from 1% to 5%, of this compound.

The administration of this pharmaceutical composition can be carried out orally, topically, ocularly, intraocularly, intravenously, parenterally, subcutaneously, epicutaneously, intradermally, transdermally, intramuscularly or enterally, by rectal, intranasal, sublingual, buccal or intrarespiratory administration or by nasal inhalation. It is preferable for the pharmaceutical composition to be a dermatological composition administered topically to the skin, the mucous membranes and/or the skin appendages.

As a variant, the hydroperoxy alcohol compound, or a 1,2,4-trioxane thereof, can be used as an antimicrobial agent for cleansing, sanitizing, disinfecting and/or sterilizing an inert surface, that is to say a surface other than the skin, the skin appendages and the mucous membranes, in particular a hard surface or a textile.

In any event, the hydroperoxy alcohol used according to the invention, or a 1,2,4-trioxane thereof, can be included in a composition which also comprises one or more excipients. The composition may comprise, in certain cases, a combination of at least one hydroperoxy alcohol compound and at least one 1,2,4-trioxane.

The excipient(s) of the composition may be any excipient known to those skilled in the art and suitable for the type of application of the composition. Examples of excipients are in particular sequestrants, antioxidants, preservatives, fillers, electrolytes, humectants, dyes, fragrances, essential oils, moisturizing agents, vitamins, essential fatty acids, surfactants and/or emulsifiers, gelling agents, thickeners, buffering agents, lipophilic excipients, or a mixture thereof. Of course, those skilled in the art will take care to choose this or these optional excipients, and/or the amount thereof, in such a way that the advantageous properties of the composition used according to the invention are not, or not substantially, impaired.

A subject of the invention thus relates to a composition containing an effective amount of at least one hydroperoxy alcohol compound, or a 1,2,4-trioxane thereof, and at least one excipient chosen from surfactants, thickeners, pigments, dyes, fragrances and mixtures thereof, in particular a cosmetic or dermatological composition also comprising a physiologically acceptable medium optionally containing water.

In the case where it is a cosmetic or dermatological composition, the composition according to the invention may also comprise at least one active agent chosen from desquamating agents, calmatives or anti-inflammatories, anti-seborrheic agents and mixtures thereof. As a variant or in addition, the composition may comprise at least one active agent chosen from probiotics, such as a bacterium of the Lactobacillus or Bifidobacterium genus; prebiotics, such as beta-glucan, inulin, α-galactooligosaccharides and fructooligosaccharides; postbiotics, such as lactic acid and polyunsaturated fatty acids of omega-3 and omega-6 type; and mixtures thereof. In the case where it constitutes a detergent composition, the composition according to the invention generally comprises water and at least one surfactant chosen from anionic, cationic, amphoteric and nonionic surfactants and mixtures thereof.

### EXAMPLES

The invention will be understood more clearly in the light of the following examples, which are given purely by way of illustration and the purpose of which is not to limit the scope of the invention, defined by the appended claims.

### Example 1: Preparation of the Oleic Acid-Derived Hydroperoxy Alcohol

4.52 mmol of 9,10-epoxystearic acid, 4.27 μmol of phosphotungstic acid, 1.0 molar eq. of an aqueous solution of hydrogen peroxide at 30% m/V and 7.8 ml of tert-butanol are introduced into a 25 ml round-bottomed flask equipped with a magnetic bar. The mixture is stirred at 50° C. at 900 revolutions/min for 60 minutes. At the end of the stirring, a ¹H NMR analysis of the reaction crude diluted in 0.5 mL of deuterated chloroform (CDCl₃) shows that the 9,10-epoxystearic acid is converted (conversion>99%) into a mixture of 9(10)-hydroperoxy-10(9)-hydroxystearic acid (HPA) with a yield of 81%. 9,10-dihydroxystearic acid is also obtained with a yield of 16%.

### Example 2: Antimicrobial Activity of the Oleic Acid-Derived Hydroperoxy Alcohol (or “HPA”)

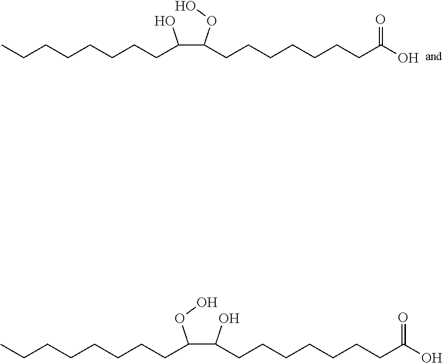 [SMILES: CCCCCCCCC(O)C(CCCCCCCC(=O)O)OO ; CCCCCCCCC(OO)C(O)CCCCCCCC(=O)O]

The tests were carried out at the surface: a solution of sample at 10 000 ppm in DMSO for a sample of HPA at 72% purity (i.e. 15 000 ppm of active material) was dried on an inert support. Once the solution had dried, a volume of inoculum of each bacterial strain was placed on the support in contact with the sample for 0 and 24 h at ambient temperature.

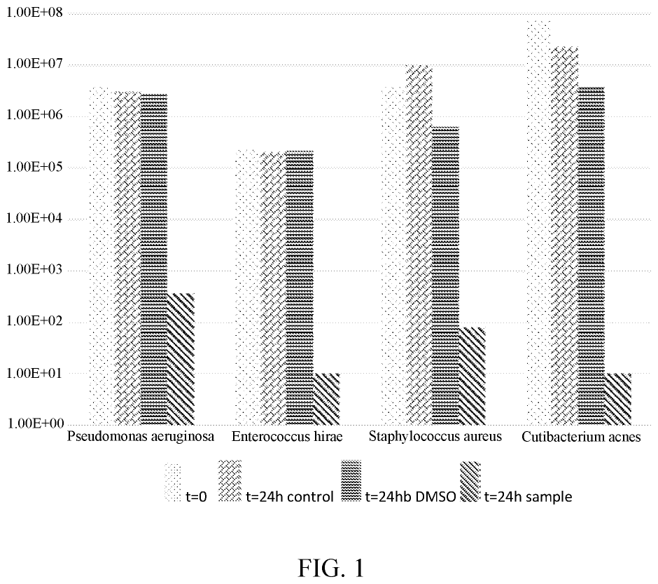

In order to verify the nontoxicity of the DMSO, a pure solution of DMSO was dried on the support under the same conditions as the samples. A nontreated support (noted support control in FIG. 1) was also inoculated, in order to verify the stability of the strains under the conditions of the test.

In order to quantify the contamination at each measurement time, a count was performed by spreading out, at the surface or in the mass, of decimal dilutions of the sample on TSA agar or Columbia agar (for C. acnes). The results were expressed in “colony-forming units” (CFUs) on the support.

The Bacterial Strains Targeted are:

Gram−: Pseudomonas aeruginosa

Gram+: Cutibacterium acnes, Enterococcus hirae, Staphylococcus aureus

FIG. 1 demonstrates that the hydroperoxy alcohol of oleic acid has antibacterial activity, in particular:
bactericidal activity on P. aeruginosa: after 24 h of contact, a logarithmic reduction in the population of approximately 4 log 10 relative to t=0 is observed.
a high bactericidal activity on E hirae: after 24 h of contact, a logarithmic reduction of the population of approximately 5 log 10 relative to t=0 is observed.
a high bactericidal activity on S. aureus: after 24 h of contact, a logarithmic reduction of the population of approximately 5 log 10 relative to t=0 is observed.
a high bactericidal activity on C. acnes: after 24 h of contact, a logarithmic reduction of the population of approximately 6 log 10 relative to t=0 is observed.

### Example 3: Evaluation of the Cytotoxicity of Hydroperoxy Alcohols (or “HPA”) According to the Invention

Normal human dermal fibroblasts were cultured in monolayer form in a DMEM medium (1% FCS) for 24 h in the presence of a solution containing various concentrations of an olive oil-derived HPA.

This HPA was prepared according to a process similar to that described in example 1 which was carried out on olive oil that had been previously epoxidized in a manner conventional for those skilled in the art.

Another series of tests was carried out on normal human epidermal keratinocytes cultured in a serum-free medium without epidermal growth factors.

Cell viability was demonstrated by colorimetry by means of a detection kit using the WST-8 tetrazolium salt and measures the absorbance at 450 nm, by comparison with a nontreated control. Three tests were carried out for each concentration tested, and also for the control. The results of these tests are collated in the table below:

Mean cell viability (%)

| | Test on fibroblasts | Test on keratinocytes |
|---|---|---|
| 0.0004% | 95 | 98 |
| 0.001% | 98 | 101 |
| 0.004% | 98 | 102 |
| 0.011% | 99 | 108 |
| 0.033% | 96 | 115 |
| 0.1% | 102 | 97 |
| Control | 100* | 100** |

*varies from 97 to 103%;
**varies from 78 to 121%

As emerges from this table, the compound according to the invention exhibits good innocuity with respect to the cells of the skin over a wide range of concentrations and can thus be used for cosmetic and dermatological applications.

### Example 4: Shampoo

The ingredients below, identified in uppercase letters by their INCI names, can be mixed conventionally by those skilled in the art in order to obtain a foaming shampoo:

| | |
|---|---|
| SODIUM C14-16 OLEFIN SULFONATE | 9.20% |
| COCO BETAINE | 2.50% |
| COCO GLUCOSIDE | 2.50% |
| POLYQUATERNIUM-10 | 0.50% |
| DIMETHICONE | 1.75% |
| CARBOMER | 0.25% |
| GLYCOL DISTEARATE | 1.20% |
| HPA from olive oil | 0.50% |
| SALICYLIC ACID | 0.20% |
| Fragrance base* | 0.20% |
| SODIUM CHLORIDE | qs |
| HEXYLENE GLYCOL | qs |
| SODIUM HYDROXIDE | qs |
| CITRIC ACID | qs |
| Water | qs 100% |

*containing citral, geraniol, linalool, nerol, α-terpineol, citronellol, citronellal, thymol, eugenol and β-pinene.

1-11. (canceled)

12. A method of improving the antimicrobial properties of at least one preservative comprising contacting a preservative with a hydroperoxy alcohol compound or a 1,2,4-trioxane thereof.

13. A method of reducing at least one unattractive manifestation of the imbalance of the skin microbiota comprising contacting the skin of a subject with a hydroperoxy alcohol compound or a 1,2,4-trioxane thereof.

14. The method of claim 13, wherein said hydroperoxy alcohol compound has the formula (Ia) and/or (Ib):
R¹—CR⁴(OOH)—CR³(OH)—R² (Ia)
R²—CR³(OOH)—CR⁴(OH)—R¹ (Ib)
wherein:
R¹, R² R³ and R⁴ each independently represent a hydrogen atom or a saturated, monounsaturated or polyunsaturated alkyl group or an aryl group, which is optionally substituted, containing from 1 to 30 carbon atoms or a group —L—A where L is a bond or an optionally substituted, linear or branched alkylene chain containing from 1 to 30 carbon atoms, and A represents a hydrogen atom or a group —COXR′ wherein X denotes an oxygen atom or a group —NR″, and R′ and R″ each independently denote a group selected from: a hydrogen atom, a C₁-C₈ alkyl group, a C₄-C₈ aryl group or a —(CH₂)—CH(OCOR⁵)—CHOCOR⁶ group, R⁵ and R⁶ each independently denoting a linear or branched C₈-C₂₂ alkyl group which is optionally substituted and/or interrupted with at least one hydroperoxy and/or hydroxyl group,
or else R¹ and R², or R³ and R⁴, or R¹ and R⁴, or R² and R³ together form an optionally substituted carbocycle consisting of from 6 to 12 ring members.

15. The method of claim 13, wherein said 1,2,4-trioxane is at least one compound having the formula (IIa) and/or (Ib): 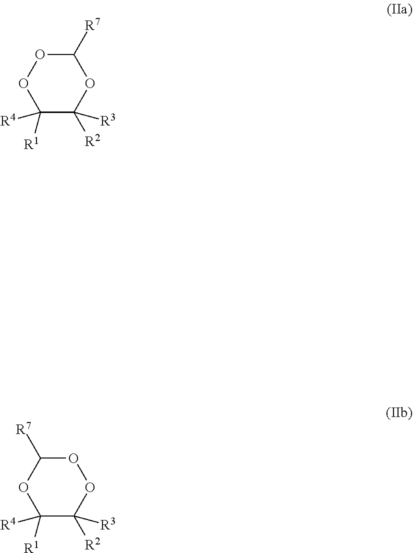
wherein:
R¹, R² R³ and R⁴ each independently represent a hydrogen atom or a saturated, monounsaturated or polyunsaturated alkyl group or an aryl group, which is optionally substituted, containing from 1 to 30 carbon atoms or a group —L—A where L is a bond or an optionally substituted, linear or branched alkylene chain containing from 1 to 30 carbon atoms, and A represents a hydrogen atom or a group —COXR′ wherein X denotes an oxygen atom or a group —NR″, and R′ and R″ each independently denote a group selected from: a hydrogen atom, a C₁-C₈ alkyl group, a C₄-C₈ aryl group or a —(CH₂)—CH(OCOR⁵)—CHOCOR⁶ group, R⁵ and R⁶ each independently denoting a linear or branched C₅-C₂₂ alkyl group which is optionally substituted and/or interrupted with at least one hydroperoxy and/or hydroxyl group,
or else R¹ and R², or R³ and R⁴, or R¹ and R⁴, or R² and R³ together form an optionally substituted carbocycle consisting of from 6 to 12 ring members, and
R⁷ represents a hydrogen atom or a saturated, monounsaturated or polyunsaturated alkyl group or an aryl group, which is optionally substituted, containing from 1 to 30 carbon atoms.

16. The method of claim 13, wherein said hydroperoxy alcohol compound or a 1,2,4-trioxane thereof is formed from an alkene selected from at least one monounsaturated or polyunsaturated fatty acid or the ester thereof, or mixtures thereof, or at least one alkyl ester of said fatty acid or at least one glyceride of said fatty acid or a terpene.

17. The method of claim 13, wherein said hydroperoxy alcohol compound is obtained by perhydrolysis of an epoxidized plant oil or olive oil.

18. The method of claim 13, wherein said hydroperoxy alcohol compound is obtained by a process comprising a step of perhydrolysis of an epoxide by reaction with an aqueous solution of hydrogen peroxide in the presence of a catalyst consisting of phosphotungstic acid.

19. The method of claim 13, wherein the unattractive manifestation is dandruff, squames, the formation of blackheads, roughness of the skin, nonuniformity of the complexion, the development of odor, and/or hair loss.

20. A method of cleansing, sanitizing, disinfecting and/or sterilizing a hard surface or textile comprising contacting the hard surface or textile with a hydroperoxy alcohol compound or a 1,2,4-trioxane thereof.

21. A method of treating a human or animal for a disease affecting the skin, the mucous membrane and/or the skin appendages of the human or animal comprising contacting the skin, the mucous membrane and/or the skin appendages of the human or animal with a hydroperoxy alcohol compound having the formula (Ia) and/or (Ib):
R¹—CR⁴(OOH)—CR³(OH)—R² (Ia)
R²—CR⁴(OOH)—CR³(OH)—R² (Ib)
wherein:
R¹, R² R³ and R⁴ each independently represent a hydrogen atom or a saturated, monounsaturated or polyunsaturated alkyl group or an aryl group, which is optionally substituted, containing from 1 to 30 carbon atoms or a group —L—A where L is a bond or an optionally substituted, linear or branched alkylene chain containing from 1 to 30 carbon atoms, and A represents a hydrogen atom or a group —COXR′ wherein X denotes an oxygen atom or a group —NR″, and R′ and R″ each independently denote a group selected from: a hydrogen atom, a C₁-C₈ alkyl group, a C₄-C₈ aryl group or a —(CH₂)—CH(OCOR⁵)—CHOCOR⁶ group, R⁵ and R⁶ each independently denoting a linear or branched C₈-C₂₂ alkyl group which is optionally substituted and/or interrupted with at least one hydroperoxy and/or hydroxyl group,
or else R¹ and R², or R³ and R⁴, or R¹ and R⁴, or R² and R³ together form an optionally substituted carbocycle consisting of from 6 to 12 ring members,
or a 1,2,4-trioxane thereof.

22. The method of claim 21, wherein the disease affecting the skin, the mucous membrane and/or the skin appendages of the human or animal is acne, herpes, mycosis, athlete's foot, chickenpox, warts, shingles, psoriasis, conjunctivitis, styes, eczema, vitiligo, atopic dermatitis or a keratosis.

23. A dermatologic or cosmetic composition containing an effective amount of at least one hydroperoxy alcohol compound, or a 1,2,4-trioxane thereof, and at least one excipient selected from surfactants, thickeners, pigments, dyes, fragrances and mixtures thereof.

24. The composition of claim 23, wherein the cosmetic or dermatological composition also comprises at least one active agent selected from the group consisting of desquamating agents, calmatives or anti-inflammatories, antiseborrheic agents, probiotic agents, prebiotic agents, postbiotic agents and mixtures thereof.
